(19) United States
(12) Patent Application Publication
(10) Pub. No.: US 2025/0381381 A1
(43) Pub. Date: Dec. 18, 2025

(54) DRESSING FOR PROTECTING SKIN ENTRY SITE OF VASCULAR ACCESS CATHETER

(71) Applicant: Cardiac Assist Holdings, LLC, Ann Arbor, MI (US)

(72) Inventors: Allen Kantrowitz, Ann Arbor, MI (US); In Ki Mun, Ann Arbor, MI (US)

(73) Assignee: Cardiac Assist Holdings, LLC, Ann Arbor, MI (US)

(21) Appl. No.: 19/240,160

(22) Filed: Jun. 17, 2025

Related U.S. Application Data
(60) Provisional application No. 63/660,607, filed on Jun. 17, 2024.
(60) Provisional application No. 63/728,200, filed on Dec. 5, 2024.

(51) Int. Cl. A61M 39/02; A61B 5/00

(52) U.S. Cl. CPC A61M 39/0247; A61B 5/4848; A61M 2039/0258; A61M 2039/0267; A61M 2039/0273; A61M 2039/0288

(57) ABSTRACT

A dressing for protecting a vascular access catheter skin entry site including a foundation dressing module and a cover dressing module. The foundation dressing module includes a single or multi-part frame defining an aperture. The frame has a foundation dressing skin-facing surface and a foundation dressing opposing surface in opposition to the foundation dressing skin-facing surface. The cover dressing module includes a cover dressing skin-facing surface and a cover dressing non-skin facing surface, a hydroscopic pad on the cover dressing skin-facing surface adapted to area-fill the aperture. The cover dressing skin-facing surface is configured to be superimposable onto the foundation dressing opposing surface bringing the hydroscopic pad into direct fluid communication with a skin entry site of the vascular access catheter.

### RELATED APPLICATIONS

This application claims priority benefit of U.S. Provisional Application Ser. No. 63/660,607, filed Jun. 17, 2024, and U.S. Provisional Application Ser. No. 63/728,200, filed Dec. 5, 2024, the contents of which are hereby incorporated by reference.

### FIELD OF THE INVENTION

The present invention in general relates to medical devices and medical dressings for protecting entry sites for vascular access catheters, and in particular to medical devices and medical dressings to protect the percutaneous entry sites of vascular access catheters from mechanical and microbiologic challenges.

### BACKGROUND OF THE INVENTION

Placement of a percutaneous vascular access catheter is a commonly performed medical procedure. Skin entry sites for percutaneous vascular access catheters create clinical susceptibility to bacterial growth and infection by acting as an ingress situs for microorganisms, leading to overgrowth, infection at the site of insertion and/or along the surface of the catheter and subsequent biofilm formation. Infection of the catheter hub and catheter-related bloodstream infections are major complications for patients with indwelling catheters (e.g., Safdar and Maki, Intensive Care Med. 2004 January; 30 (1): 62-7; Saint et al., Infect Control Hosp Epidemiol. 2000 June; 21 (6): 375-80). The aforementioned infections are commonly referred to as a skin entrance (or exit) site infection (SESI) and are common complications associated with medical treatment modalities that require a catheter to penetrate the skin. SESIs have been associated with (1) cellulitis of subcutaneous tissues; (2) erosion of tissues adjacent to skin exit site; (3) infection along the catheter to deeper planes; and (4) intractable infection and systemic sepsis. SESI is a leading cause of increased hospitalizations and morbidity for patients with indwelling catheters and in particular for patients with indwelling catheters at non-planar body locations illustratively including jointed locations such as the inside of the elbow or the back of the hand. Indwelling catheters at non-planar body locations present unique challenges in protecting the skin entry site from repeated trauma due to the higher movement frequency characteristic of non-planar body locations which can create an increased risk of infection.

At the time of placement of a vascular catheter, the skin entry site can be understood to have suffered a highly localized iatrogenic injury of the epidermis, dermis, subcutaneous tissue, local tissue microscopic vessels associated with these tissue layers and, ultimately, the walls and lumen of the catheter's target location. Contemporaneous with iatrogenic injury, a complex series of events is triggered in a cascade of the body's natural wound healing processes: Initially, in the “Hemostatic” phase of wound healing, vessel wall constituents, exposed by the local trauma, initiate vessel constriction and form peri-injury blood clots to limit blood loss. In succeeding overlapping “Inflammatory” and “Proliferative” phases, wound repair mechanisms lead to the local accumulation of body fluids, signaling molecules, proteins, tissue debris, and other biologic factors-collectively referred to as “wound exudate”-within the wound environment illustratively including the vicinity of the outer surface of the catheter and the associated tunnel through the subcutaneous tissues and the epidermal/dermal entry site, herein referred to as the “wound bed tunnel gap”. Even as the wound exudate creates a fluid communication pathway between the microbial ecosystem of the skin, the deeper tissues adjacent to the outer surface of the vascular access catheter, and the blood stream, immune system cells engulf debris and bacteria in the service of debriding the wound bed tunnel gap. Sub-acutely, after the initial tissue injury associated with the implantation of the vascular access catheter, the body attempts to repair the defect in epidermal, dermal and subcutaneous tissue layers in proliferative and migratory phases of wound healing characterized by the accumulation of activated fibroblasts, deposition of molecules such as procollagen to repair the extracellular matrix, recruitment of new vascular tissues, and creation of so-called granulation tissue in the wound bed tunnel gap.

The continued presence of the vascular access catheter prevents the cascade of wound healing processes from fully repairing the entrance site wound and interferes with the re-establishment of local skin barriers to microbiological invasion. Furthermore, the local accumulation of wound exudate creates a biological milieu that supports microbiologic growth and colonization of the regions adjacent to the surface of the catheter within the wound bed tunnel gap. Yet furthermore, such microbiologic overgrowth can serve as a staging reservoir, positioned deep to epidermal/dermal barrier defenses, from which repeated microbiologic invasive forays can be launched yet deeper into the body. These microbiologic invasions deeper into the body, beyond the immediate vicinity of the wound bed tunnel gap associated with the vascular access catheter, can result in tissue cellulitis as well as distant spread to remote sites via bloodborne, lymphatic, and/or direct pathways with subsequent bacterial endocarditis, brain abscess, epidural spinal abscess, osteomyelitis, pneumonia, thoracic empyema, and other serious life-threatening infections.

Principals of clinical practice, commonly thought to reduce risk of infectious complications associated with use of percutaneous vascular catheter access, include: (1) mechanical exclusion, from the catheter entry site, of entry of exogenous soilage; (2) mechanical stabilization of the extracorporeal segment of catheter to minimize mechanical transmission of repeated longitudinal, azimuthal, and torsional stresses via the catheter to the tissues immediately surrounding the skin, subcutaneous tissues and vessel wall adjacent to the catheter entry site, and wound bed tunnel gap; (3) optionally (and controversially), administration of topical antibiotics to the vicinity of the catheter entry site and the wound bed tunnel gap; and (4) hydrostatic draw of wound exudate fluid from the region in the wound bed tunnel gap immediately surrounding the skin, subcutaneous tissues, vessel wall adjacent to the catheter entry site, and wound bed tunnel gap.

With regards to (1) mechanical exclusion, from the catheter entry site, of entry of exogenous soilage: legacy adhesive tape & gauze ad hoc dressings are recently being superseded by carefully engineered dressings which address design issues such as adhesivity, water vapor permeability, and ability to inspect the skin adjacent to catheter entry site. A 3M TegedermÔ 1626W Transparent Film Dressing is a typical example of a commercially available transparent dressing.

With regards to (2) mechanical stabilization of the extracorporeal segment of catheter to minimize mechanical transmission of repeated longitudinal, azimuthal, and torsional stress, with resultant repetitive micromechanical tissue injury produced by mechanical action of the catheter to the tissues immediately surrounding the skin, subcutaneous tissues and vessel wall adjacent to the catheter entry site and wound bed tunnel gap: various approaches have been developed to mechanically secure the geometric relationship between the extra-corporeal segment of the catheter and the skin entrance site. Examples of commercially available catheter securement devices include Becton Dickinson Nexiva™ Closed IV Catheter System, Becton Dickinson StatLock™, and InterRad Medical SecureACath™.

With regards to (3) optional administration of topical antibiotics or antiseptics to the region of the catheter entry site and the wound bed tunnel gap: some commercially available dressings, as exemplified by Chlorhexidine-impregnated dressings such as the 3M Tegederm™ 1665, provide a means for local delivery of antibiotics and/or antiseptics to the microenvironment adjacent to the catheter entry site. Another approach relies on antibiotic molecules incorporated/impregnated into the polymeric material making up the catheter walls. Clinical controversy of the role of such topical antibiotic design attributes exists, however, due to clinical difficulties created by allergic reactions to the antiseptic/antibiotic agents and due to the threat of biologic selection of antibiotic resistant organisms with subsequent increase in yet more dangerous clinical infectious complications.

With regards to (4) hydrostatic draw of wound exudative fluid from the region immediately surrounding the skin, subcutaneous tissues, vessel wall adjacent to the catheter entry site, and wound bed tunnel gap: many successful modern wound management methods have recognized the importance of appropriate management of accumulation of wound exudative fluids. Said fluids, exudating from recently injured tissues, contain blood, water, electrolytes, glucose, proteins, formed cellular elements, and cellular debris. This complex aqueous mixture provides a rich growth media supportive of exuberant growth of microbiota. The continued presence of the vascular catheter maintains a channel defect, crossing otherwise intact skin barriers. As a result, microbial flora present on the skin can travel in the aqueous fluid communication channel created by the column of wound exudate between the skin microenvironment and the microenvironment of the peri-catheter tissues. Successful wound management methods frequently include a means to manage accumulation, within the wound area, of wound exudative fluid. Another benefit of hydrostatic draw is that it can reduce or eliminate the need for antibiotics or antiseptics due to the reduction or elimination of moisture accumulation from wound exudate. This is particularly beneficial to patients with chlorhexidine sensitivity. In simplest form, simple absorptive gauze in fluid communication with the accumulating wound exudate can draw said fluid by, for example, capillary mechanisms. Other exemplary means of creating hydrostatic draw include hydroscopic/hydrophilic dressings as exemplified by Advadraw (Advancis), Vacutex (Protex), Allevyn (Smith & Nephew), Biatain (Coloplast), Granuflex (ConvaTec), and NU DERM (Systagenix). Fibrous alternatives that resemble alginates and are not occlusive have also been developed as exemplified by Aquacel (ConvaTec).

Each of the above-described means of drawing wound exudative fluid from the wound bed tunnel gap are subject to progressive limitation of draw strength due to progressive saturation of the draw mechanism: as the draw mechanism becomes saturated, the rate of fluid draw systematically reduces and, counter-therapeutically, wound exudate and bioburden accumulate in the wound bed tunnel gap. The draw strength may be renewable, for example, by replacing the draw mechanism with a fresh new draw mechanism. By way of illustrative example, saturated gauze can be replaced by de-adhering from the skin, the adhesive tape maintaining the position of the saturated gauze, positioning fresh unsaturated gauze over the catheter exit site and then re-adhering adhesive tape to the skin to maintain the position of the gauze. Similarly, a saturated hydroscopic/hydrophilic pad can be replaced by de-adhering the associated adhesive film, maintaining the position of the saturated gel pad relative to the skin, positioning a fresh unsaturated gel pad over the catheter exit site, and then re-adhering adhesive film to the skin to maintain the position of the fresh unsaturated gel pad.

The Centers for Disease Control have summarized these efforts and synthesized clinical recommendations in the form of practice guidelines entitled Updated Recommendations on C-I Dressings—Updated Recommendations on the Use of Chlorhexidine-Impregnated Dressings for Prevention of Intravascular Catheter-Related Infections (2017).

One technique for appropriate management of wound exudative fluids in the service of promoting natural healing processes, particularly, but not exclusively during the proliferate phase, is known as negative pressure wound therapy (NPWT). Application of a reduced pressure, e.g., sub-atmospheric, to a localized zone adjacent to a wound has been found to assist in managing the accumulation of wound exudative fluid. Also, a reduced pressure may aid wound management by several other mechanisms including, but not limited to, gentle compression of tissues adjacent to the wound, improved tissue capillary flow, and other mechanisms which may aid in wound healing and inhibit bacterial growth. This technique has proven effective for chronic or non-healing wounds but has also been used for other purposes such as post-operative wound care and wound care of entry sites of implantable medical devices as exemplified in U.S. Pat. No. 11,197,988 of Viaderm LLC that provides vacuum-assisted dressing optimized for placement at the percutaneous entrance site of some percutaneous medical appliances such as Peritoneal Dialysis catheters. Interestingly, NPWT dressing methods can be understood in similar terms as detailed above with regards to continued maintenance of fluid draw pressure in that the mechanically-driven NPWT dressing maintains the fluid draw pressure by mechanical means, albeit in a manner which is generally regarded as too cost-prohibitive to be used in the clinical setting of short-term vascular catheter access sites anticipated in this invention.

Use of a vacuum assisted dressing, as exemplified by the Viaderm Clarity™ dressing described in US20220079509, is believed to be clinically useful in mitigating infections associated with the entry site of medical appliances crossing the skin. However, a clinical concern arises in considering the use of such vacuum-assisted dressings to mitigate entry site infections associated with those medical appliances which are in direct communication with the lumen of a blood vessel, Cerebrospinal Fluid (CSF)-containing anatomic site, or other body fluid containing anatomic site from which major unplanned escape of body fluid via an inadvertently-created pathway external to the catheter and into the vacuum mechanism is possible. Safe use of a vacuum-assisted dressing to treat such a catheter entry site would beneficially include a means to detect, interrupt, and/or limit said inadvertent escape of clinically significant volumes of, by way of illustrative example, blood or CSF, into the vacuum channels and vacuum mechanism of said vacuum-assisted dressing. However, such a means to detect, interrupt and/or limit the inadvertent escape of clinically significant volumes of body fluids would greatly increase the cost and complexity of the dressing.

The cost and complexity of incorporation, into a skin entry site dressing, of a means to detect, interrupt, and/or limit the inadvertent escape of clinically significant volumes of body fluids may be advantageously avoided by the use of a hydroscopic/hydrophilic gel pad dressing including features as described hereinabove. Representative hydroscopic/hydrophilic dressing features include a means to manage accumulation within the wound area of wound exudative fluid. In simplest form, absorptive gauze in fluid communication with the accumulating wound exudate can draw said fluid by, for example, capillary mechanisms. Other means of creating hydrostatic draw include hydroscopic dressings as exemplified by Advadraw (Advancis), Vacutex (Protex), Allevyn (Smith & Nephew), Biatain (Coloplast), Granuflex (ConvaTec) and NU DERM (Systagenix). Fibrous alternatives that resemble alginates and are not occlusive have also been developed exemplified by Aquacel (ConvaTec). As mentioned hereinabove in paragraph 10, a well-known limitation of such hydroscopic/hydrophilic design features is that the hydroscopic/hydrophilic design features have only a limited volumetric capacity. Standard recommendations for use of such dressings emphasize the importance of frequent bedside assessment of the saturation state of the hydroscopic/hydrophilic design feature. For example, once per day nurses are instructed to press their finger on to the external surface of hydrogel pad through the outside plastic film of the dressing so as to attempt to push fluid from the hydrogel pad, thereby assessing capacity of the hydrogel pad to continue to create hydrostatic draw pressure in the service of continued draw of wound exudate fluid. Nurses are encouraged to replace the hydrogel pad on the day that the fluid displacement test indicates that the pad is saturated. One drawback of frequent replacement of prior-art saturated adhesive-backed hydrogel pads with new fresh prior-art adhesive-backed hydrogel pads is that it subjects the patient to multiple epidermis adhesion-de-adhesion dressing change cycles and can raise concern for mechanical irritation or injury to the patient's skin due to their attendant repetitive de-epithelization injuries. Hence, as commonly employed, the hydrogel pad provides an interrupted, episodic, rather than continuous, hydrostatic draw upon the wound exudative fluid. Unfortunately, the periodicity of the hydrostatic draw implies that the microbial population will be episodically provided with a surfeit of wound exudate fluid that will predictably provide an overabundance of microbial-growth-promoting substances on an intermittent schedule. Notably, prior-art dressing designs force medical care givers to confront a therapeutic dilemma balancing the need to maintain a vigorous and active fluid draw in the vicinity of the wound bed tunnel gap and catheter skin entrance site against the need to minimize skin irritation caused by repeated cycles of skin adhesive film adherence and de-adherence cycles and also against the need to conveniently and cost-effectively clinically reassess the saturation status of the fluid draw mechanism.

Hence, with commonly available prior art vascular access site dressings incorporating hydroscopic/hydrophilic design features, clinically appropriate dressing management methods attempt to advantageously balance the counter-therapeutic effect of over-accumulation of wound exudate fluid adjacent to the skin entry site against the counter-therapeutic effect, on the skin, of frequent adhesion-de-adhesion events with their attendant de-epithelization injuries and the cost and complexity of frequent reassessments of the saturation status of the fluid draw mechanism. An illustrative example of prior art dressing management is the use of Montgomery straps in surgical settings. Montgomery straps integrate paired adhesive straps applied to either side of a wound (usually abdominal). The central sections of the paired adhesive straps are folded back onto themselves with several perforations at the leading edges. This arrangement provides a method for securing a bandage against the skin and subsequently changing it without having to replace adhesive tape with each change cycle.

Thus, there is a need for a wound dressing design-optimized for clinical application to the skin entry site of a vascular access catheter which allows a caregiver to rapidly, conveniently, and cost-effectively detect loss of hydrostatic draw, as exemplified by fluid saturation of a hydroscopic/hydrophilic gel pad, and easily and rapidly restore the hydrostatic draw mechanism, for example by replacing a hydroscopic/hydrophilic gel pad perhaps more frequently than once per day, without subjecting the patient's skin to repetitive de-epithelization injury. There is a further need for a design-optimized wound dressing that provides for mechanical stabilization of a vascular access catheter during clinical use and renewal of a hydrostatic draw mechanism for example, by exchange of a hydroscopic/hydrophilic gel pad. A need also exists for a design-optimized wound dressing that optionally allows inclusion of antibiotics and/or antiseptics, by way of example, within a hydroscopic/hydrophilic gel pad or by some other method of topical administration, in the service of influencing the ecology of the microbial population in the vicinity of the skin entrance site of the vascular access catheter. There is a still further need for an effective wound management system to mitigate infection of such wounds to increase safety, improve quality of life, reduce healthcare costs, and lessen the burden of wound care on nursing staff and caregivers. This is yet a further need for a design-optimized wound dressing for protecting a vascular access catheter skin entry site at a non-planar body location illustratively including joint locations such as the back of the knee or the inside of the elbow. Due to the higher frequency of stress forces at non-planar body locations, a design-optimized wound dressing that is longitudinally, latitudinally, azimuthally, and torsionally flexible is beneficial in preventing additional damage and additional exudate accumulation in and around the skin entry site of a vascular access catheter.

### SUMMARY OF THE INVENTION

The present invention provides a dressing for protecting a vascular access catheter skin entry site. The dressing includes a foundation dressing module and a cover dressing module. The foundation dressing module includes a frame defining an aperture. The frame has a foundation dressing skin-facing surface and a foundation dressing opposing surface in opposition to the foundation dressing skin-facing surface. The cover dressing module includes a cover dressing skin-facing surface and a cover dressing non-skin facing surface, a hydroscopic pad on the cover dressing skin-facing surface adapted to area-fill the aperture. The cover dressing skin-facing surface is configured to be superimposable onto the foundation dressing opposing surface bringing the hydroscopic pad into direct fluid communication with a skin entry site of the vascular access catheter.

The present invention additionally provides a dressing for protecting a vascular access catheter skin entry site at a non-planar body location. The dressing includes a cover dressing module as described above and a foundation dressing module that includes a multi-part frame defining a groove. The multi-part frame having a foundation dressing skin-facing surface and a foundation dressing opposing surface opposing surface in opposition to the foundation dressing skin-facing surface. The dressing module also includes a frame extension adjacent to one part of the multi-part frame and having a top surface and a bottom surface and a securement attached to the top surface of said frame extension adapted to secure a vascular access catheter.

Additionally, the present invention provides a method of protecting an environment in and around a skin entry site of a vascular access catheter. The method includes placing an inventive dressing on skin around the skin entry site of a vascular access catheter.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIG. 1 shows a perspective view of a foundation dressing module according to certain embodiments of the present invention;

FIG. 2 shows a perspective view of a foundation dressing module with a vascular access catheter placed at a skin entry point with vascular access catheter hub placed within an inactivated securement according to certain embodiments of the present invention;

FIG. 3 shows a perspective view of a foundation dressing module with a vascular access catheter placed at a skin entry point with vascular access catheter hub placed within an activated securement according to certain embodiments of the present invention;

FIG. 4 shows a perspective view of a foundation dressing module with a vascular access catheter placed at a skin entry point with vascular access catheter hub placed within an activated securement and a cover dressing module according to certain embodiments of the present invention;

FIG. 5 shows a perspective view of a foundation dressing module with a vascular access catheter placed at a skin entry point with vascular access catheter hub placed within an activated securement and a cover dressing module partially adhered onto the foundation dressing module according to certain embodiments of the present invention;

FIG. 6 shows a perspective view of a foundation dressing module with a vascular access catheter placed at a skin entry point with vascular access catheter hub placed within an activated securement and a cover dressing module more than 50% adhered onto the foundation dressing module according to certain embodiments of the present invention;

FIG. 7 shows a perspective view of a foundation dressing module with a vascular access catheter placed at a skin entry point with vascular access catheter hub placed within an activated securement and a cover dressing module fully adhered onto the foundation dressing module according to certain embodiments of the present invention;

FIG. 8 shows a perspective view of a foundation dressing module with a multi-part frame according to embodiments of the present invention;

FIG. 9 shows a perspective view of a cover dressing module according to embodiments of the present invention;

FIG. 10 shows a perspective view of a foundation dressing module according to embodiments prior to being placed at a skin entry point;

FIG. 11 shows a perspective view of a foundation dressing module of FIG. 10 being placed at a skin entry point with the catheter not yet fixed in the dressing and with the release liner still attached to the bottom of the dressing;

FIG. 12 shows a perspective view of the foundation dressing module of FIG. 11 with the release liner removed from the bottom of the dressing to secure the dressing to the skin around the skin entry point;

FIG. 13 shows a perspective view of the foundation dressing module of FIG. 12 with the catheter loaded into the saddle of the dressing;

FIG. 14A shows a perspective view of a cover dressing module for covering a foundation dressing module prior to the release liners being removed therefrom;

FIG. 14B shows a perspective view of the cover dressing module of FIG. 14A with the release liners removed therefrom;

FIG. 15 shows a perspective view of the foundation dressing module of FIG. 13 with the catheter loaded into the saddle of the dressing and with a cover dressing module of FIG. 14B mounted thereto; and

FIG. 16 shows a perspective view of the foundation dressing module and cover dressing module of FIG. 15 with the window frame removed from the cover dressing module.

### DETAILED DESCRIPTION OF THE INVENTION

Embodiments of the present invention have utility as a wound dressing design-optimized for clinical application to the skin entry site of a vascular access catheter which allows a caregiver to detect loss of hydrostatic draw rapidly, conveniently, and cost-effectively and easily and rapidly restore the hydrostatic draw mechanism, without subjecting the patient's skin to repetitive de-epithelization injury. Embodiments of the present invention have further utility as a design-optimized wound dressing that provides for mechanical stabilization of a vascular access catheter during clinical use and renewal of a hydrostatic draw mechanism for example, by exchange of a hydroscopic gel pad. Embodiments of the present invention have still further utility as a design-optimized wound dressing that optionally allows inclusion of antibiotics and/or antiseptics, by way of example, within a hydroscopic/hydrophilic gel pad or by some other method of topical administration, in the service of influencing the ecology of the microbial population in the vicinity of the skin entrance site of the vascular access catheter. Embodiments of the present invention have yet more utility as a design-optimized wound dressing for protecting a vascular access catheter skin entry site at a non-planar body location illustratively including joint locations such as the back of the knee or the inside of the elbow. Due to the higher frequency of stress forces at non-planar body locations, a design-optimized wound dressing that is longitudinally, latitudinally, azimuthally, and torsionally flexible is particularly beneficial in preventing additional damage and additional exudate accumulation in and around the skin entry site of a vascular access catheter.

Numerical ranges cited herein are intended to recite not only the end values of such ranges, but the individual values encompassed within the range and varying in single units of the last significant figure. By way of example, a range of from 0.1 to 1.0 in arbitrary units according to the present invention also encompasses 0.2, 0.3, 0.4, 0.5, 0.6, 0.7, 0.8, and 0.9; each independently as lower and upper bounding values for the range.

The following description of various embodiments of the invention is not intended to limit the invention to these specific embodiments, but rather to enable any person skilled in the art to make and use this invention through exemplary aspects thereof.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

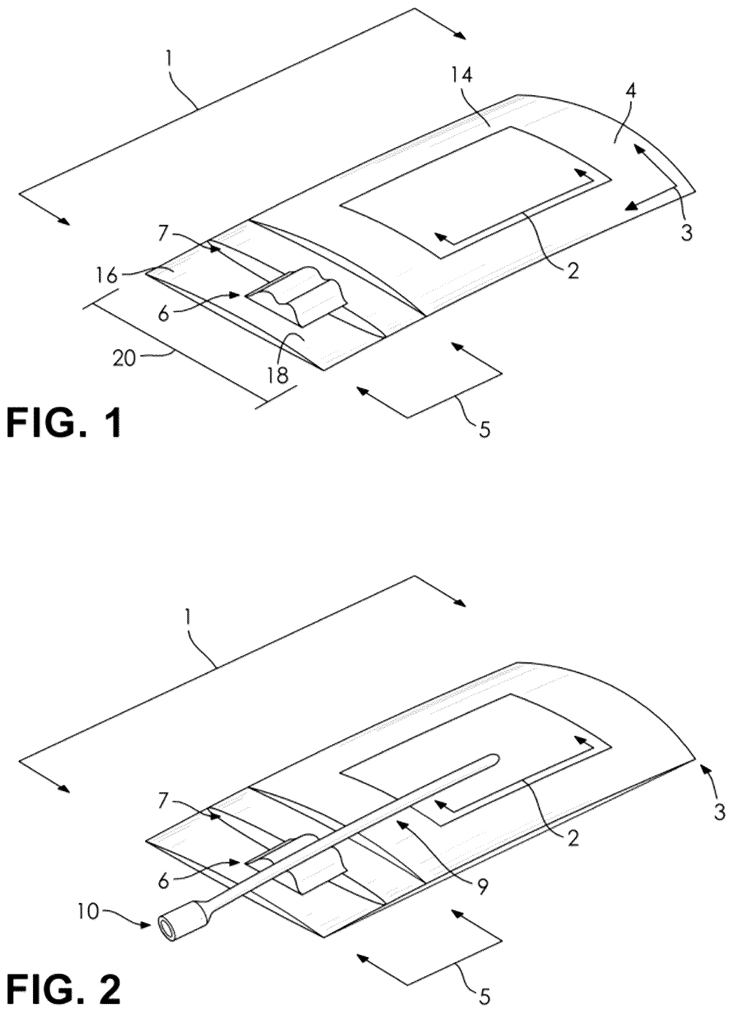

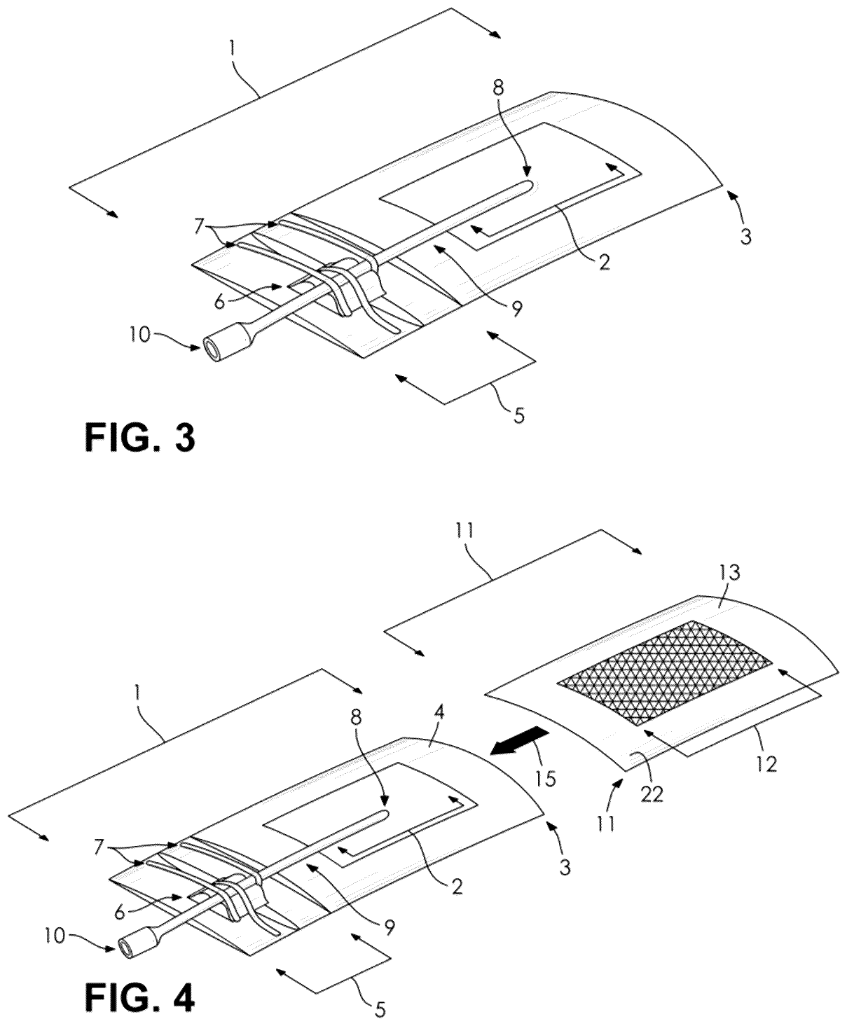

As shown in FIGS. 1-2, a foundation dressing module is shown generally at 1. The foundation dressing module 1 has a frame 3 defining an aperture 2. It is appreciated that the aperture 2 is configured to accommodate, straddle, and leave exposed an anticipated location of a skin entry site 8 of a vascular access catheter 9. The frame 3 has a foundation dressing non-skin facing surface 4 and a foundation dressing skin-facing surface 14. In some embodiments, the foundation dressing skin-facing surface 14 is backed with a skin-compatible adhesive. In other embodiments, the foundation dressing non-skin facing surface 4 has an adhesive. In still other embodiments, the foundation dressing skin-facing surface 14 is backed with a skin-compatible adhesive and the foundation dressing non-skin facing surface 4 has an adhesive. It is appreciated that after being positioned and adhered to the skin of a patient, with the aperture 2 accommodating, straddling, and leaving exposed the skin entry point 8 of the vascular access catheter 9, the foundation dressing non-skin facing surface 4 serves as a landing zone to receive the cover dressing module 11 as described in greater detail hereinbelow. A frame extension 5 adjacent to the frame 3 has a top surface 16 and bottom surface 18. In embodiments, the bottom surface 18 is backed with a skin-compatible adhesive. In some embodiments, the top surface 16 has an adhesive. In still other embodiments, the top surface 16 has an adhesive and the bottom surface 18 is backed with a skin-compatible adhesive. While the FIGs. show the aperture 2, frame 3, and frame extension 5 as rectangular, it is appreciated that a variety of other shapes and conformations are contemplated herein and illustratively include: circular, oblong, triangular, trapezoidal, quadrilateral, diamond, pentagonal, hexagonal, heptagonal, and octagonal. It is further appreciated that the frame extension 5 is a conventional component, the function of which is enhanced by usage in conjunction with a foundation dressing module according to the present invention. A securement 20 is attached to the top surface 16 of the frame extension 5. While the FIGs. show the securement 20 as having a combination saddle 6 and at least one adhesive strap 7 that can be subsequently activated as shown in FIGS. 3-7 to capture, confine, and stabilize external segments of a vascular access catheter 9 and/or the hub 10 of a vascular access catheter 9, it is appreciated that in some embodiments, additional securements 20 are contemplated in embodiments of the present invention and illustratively include buttoned straps, VELCRO®, buckles, clasps, and hinge joints. It is further appreciated that embodiments of the present invention can function using prior art securements 20 illustratively including Montgomery straps. It is further appreciated that although FIGS. 1-2 show a securement 20 which accommodates and stabilizes a single lumen catheter 8 with a single hub external port 10, it is generally recognized that current medical practice frequently involves catheters with various numbers of catheter lumens, with corresponding intermediate and terminal hub designs. As such, in some embodiments, securement 20 accommodates and stabilizes catheters with more than one catheter lumen with more than one corresponding intermediate and terminal hubs as available in clinical practice.

FIG. 4 shows the foundation dressing module 1 after it has been positioned and adhered to the skin with the aperture 2 positioned to accommodate, straddle, and leave exposed the location of the skin entry point 8 of the vascular access catheter 9. FIG. 4 also shows the hub 10 of the vascular access catheter 9 stabilized via the activated securement 20 as indicated by the position of the at least one adhesive strap 7. The cover dressing module is shown generally at 11. The cover dressing module 11 has a cover dressing skin-facing surface 13 and a cover dressing non-skin facing surface 22, and hydroscopic pad 12 on the cover dressing skin-facing surface 13. In embodiments, the hydroscopic pad 12 is sized and configured to area-fill the aperture 2. It is appreciated that in some embodiments, the cover dressing skin-facing surface 13 has an adhesive. In other embodiments, the cover dressing skin-facing surface 13 is sized and configured to be superimposable on the foundation dressing non-skin facing surface 4 of the frame 3 which acts as a landing zone to receive the cover dressing skin-facing surface 13 of the cover dressing module 11. In some embodiments, the inventive modular dressing is fully assembled by affixing the cover dressing module 11 onto the frame 3. It is appreciated that in embodiments, the fully assembled dressing brings the hydroscopic pad 12 into direct fluid communication with the skin entry point 8 of the vascular access catheter 9 so that reactive wound fluid emerging from the skin entry point 8 will be absorbed into the hydroscopic pad 12. The arrow identified by reference numeral 15 shows the anticipated positioning and adherence of the cover dressing module 11 onto the foundation dressing module 1. Not shown to improve the clarity of the FIGs., but included within embodiments of the invention, are a fluid saturation detector and a fluid saturation status indicator—both described in further detail hereinbelow—to convey fluid saturation information to, by way of illustrative example, medical staff, a patient, a patient's family.

Not depicted to improve the clarity of the figures but included within embodiments of the invention, are one or more release layer disposable adhesive guards, collectively covering and/or shielding adhesive layers/coatings applied to the foundation dressing skin-facing surface 14 of the frame 3, the foundation dressing non-skin facing surface 4 of the frame 3, the top surface 16 of the frame extension 5, the bottom surface 18 of the frame extension 5, the cover dressing skin-facing surface 13 of the cover dressing 11, or a combination thereof. It is appreciated that the one or more adhesive guards are applied at time of manufacture of embodiments of the present invention or at another time after manufacture in other embodiments of the present invention. It is further appreciated that after appropriate provisional positioning of the foundation dressing 1, the one or more adhesive guards are sequentially peeled away to expose, and thereby activate the adhesive layers/coatings, thereby allowing fixation of the position of the foundation dressing module 1 onto the skin, the position of the cover dressing module 11, or a combination thereof. Also not depicted to improve the clarity of the FIGs., but included within embodiments of the invention, is a first set of direction elements illustratively including tabs, extensions, geometric details, graphic details, or combinations thereof which direct the medical care giver as to the appropriate optimum order and sequence of removal of the set of peel-away disposable adhesive guards.

Not depicted to improve the clarity of the figures but included within embodiments of the present invention is a second set of directional elements illustratively including tabs, extensions, geometric details, graphic details, or a combination thereof are provided to aid in the alignment of the cover dressing module 11 with the foundation dressing module 1. In embodiments, the second set of directional elements is integrated into the cover dressing module 11. In other embodiments, the second set of directional elements is integrated into the foundation dressing module 1. In still other embodiments, the second set of directional elements is integrated into both the foundation dressing module 1 and the cover dressing module 11.

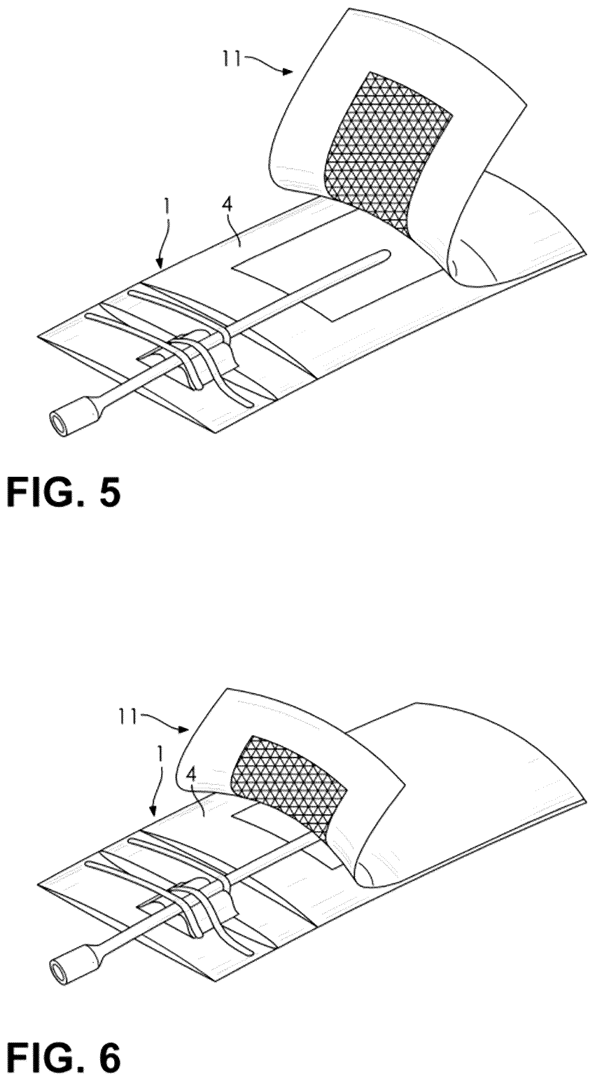

FIG. 5-6 show versions of a mid-alignment snapshot of the cover dressing module 11 as the cover dressing skin-facing surface 13 is being aligned and adhered to the frame 3 of the foundation dressing module 1.

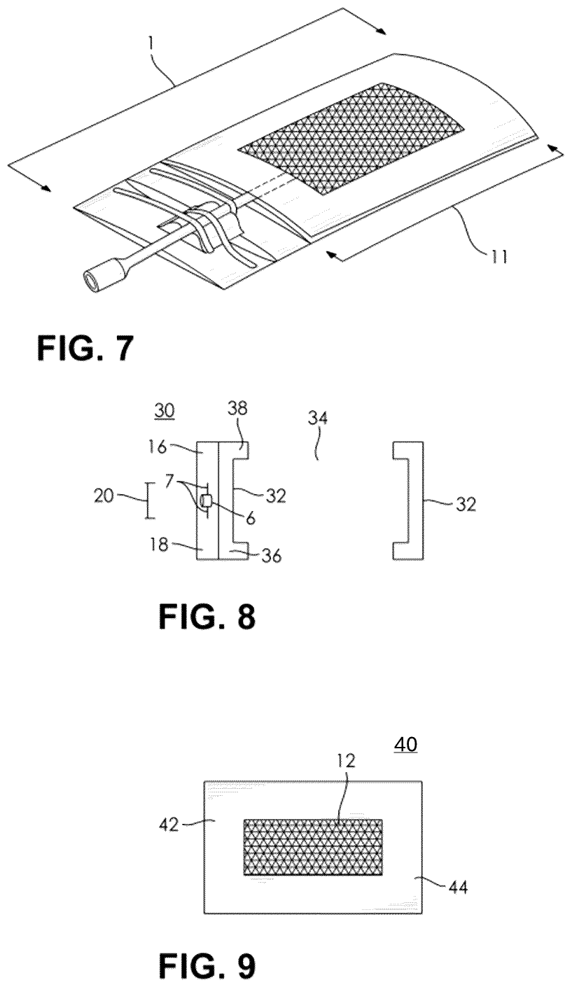

FIG. 7 shows an embodiment of a fully assembled inventive dressing module with the cover dressing module 11 in its final position with the cover dressing skin-facing surface 13 aligned and adhered to the frame 3 of the foundation dressing module 1.

FIG. 8 shows a foundation dressing module of an inventive embodiment dressing for protecting a vascular access catheter skin entry site at a non-planar body location generally at 30. It is appreciated that non-planar body locations illustratively include joints such as the back of the knee and inside of the elbow and other problematic areas such as the groin crease, knuckle, and above the clavicle. It is further appreciated that non-planar body locations render vascular access catheter skin entry sites more difficult to maintain owing to the higher frequency of stress forces caused by increased non-planar movement of such body locations. According to embodiments, the foundation dressing module 30 has a multi-part frame 32 defining a groove 34. The multi-part frame 32 has a foundation dressing non-skin facing surface 36 and a foundation dressing skin-facing surface 38. Embodiments of the present invention contemplate the multi-part frame 32 formed into a variety of shapes illustratively including an “X” shape, a “Y” shape, a “C” shaped, curved, circular, and angular. A frame extension 5 is adjacent to one part of the multi-part frame and has a top surface 16 and a bottom surface 18. A securement 20 is attached to the top surface 16 of the frame extension 5 that is configured to secure a vascular access catheter.

FIG. 9 shows a cover dressing module of an inventive embodiment dressing for protecting a vascular access catheter skin entry site a non-planar body location generally at 40. The cover dressing module has a cover dressing skin-facing surface 42 and a cover dressing non-skin facing surface 44. A hydroscopic pad 12 on the cover dressing skin-facing surface 42 is configured to area-fill the groove 34. The cover dressing skin facing surface 42 is configured to be superimposable onto the foundation dressing non-skin facing surface 36 bringing the hydroscopic pad 12 into direct fluid communication with a skin entry site 8 of the vascular access catheter.

In embodiments of the present invention, the cover dressing module 40 floats above the skin entry site 8 when attached to the foundation dressing non-skin facing surface 36. In some embodiments, it is appreciated that the cover dressing module 40 floats above the skin entry site 8 when attached to the foundation dressing non-skin facing surface 36 while also maintaining the hydroscopic pad 12 in direct fluid communication with the skin entry site 8. In other inventive embodiments, the cover dressing module 40 is longitudinally, latitudinally, azimuthally, torsionally flexible, or combination thereof. In inventive embodiments, the cover dressing 40 is formed from a folding pattern illustratively including herringbone, accordion, yoshimura, miura, waterbomb, kresling, resich, moiré, or a combination thereof. In some embodiments, the foundation dressing non-skin-facing surface 36, the foundation dressing skin-facing surface 38, the top surface 16 of the frame extension 5, the bottom surface 18 of the frame extension, the cover dressing skin-facing surface 42, or a combination thereof have an adhesive. In certain embodiments, the adhesive is skin-compatible.

While use of a hydroscopic pad 12 in embodiments of the present invention to present a hydrostatic draw force to the skin entry point 8 of a vascular access catheter 9 is detailed hereinabove, it is appreciated that other embodiments of the present invention include alternative options to present a hydrostatic draw force to the skin entry point 8 of a vascular access catheter 9. Such alternative options contemplated in embodiments of the present invention illustratively include hydrogel, absorbent gauze, super absorbent polymers, microfans, Bluetooth sensors, sheet battery to operate the aforementioned microfans and/or Bluetooth sensors, wound sniffing animals, vacuum-assisted mechanisms similar to those disclosed in US US20220079509. As used herein, a water-vapor-permeable membrane without or with local-atmospheric-facilitated evaporation of water vapor originating from the vicinity of the skin entry point 8 of the vascular access catheter 9, or a combination thereof.

Embodiments of the invention include a means of detection and signaling of the fluid saturation status and/or the remaining capacity to continue to present hydrostatic draw force to the skin entry point. By way of illustrative example, such detection and signaling means include a paper, plastic, or composite sheet/film which, when saturated with wound fluid, changes its optical or physical properties so that a visual or optical indicator signals the fluid saturation status and/or the remaining capacity to continue to present hydrostatic draw force to the skin entry point. Alternatively, an electrochemical sensor or fluid sensor is provided. These are exemplified in M. Hajnsek et al. “An electrochemical sensor for fast detection of wound infection based on myeloperoxidase activity”, Sensors and Actuators B: Chemical (2015), 209, pp. 265-274; and M. Messaoud et al. “Flexible sensors for real-time monitoring of moisture levels in wound dressings”, J. Wound Care (2018), 27 (6), pp. 385-391. Such sensors can produce a signal correlated with fluid saturation status and/or the remaining capacity to continue to present hydrostatic draw force to the skin entry point. Said signal may be recognized by a caregiver by direct inspection of the dressing and/or recognized by remote reporting means, RF or otherwise.

Other embodiments of the invention include a means of detection and signaling of the infection status of the fluids and/or tissues in the vicinity of the skin entry point. By way of illustrative example, such means could include a paper, plastic, or composite sheet/film which, when in the presence of infection in the wound fluid and/or tissues adjacent to the skin entry point, changes its optical or physical properties so that a visual or optical indicator signals the infection status of the fluids and/or tissues in the vicinity of the skin entry point. Alternatively, an electrochemical sensor, indirectly such as those of M. Hajnsek et al. or M. Messaoud et al.; or B. Mirani et al. “Smart Dual-Sensor Wound Dressing for Monitoring Cutaneous Wounds” Adv. Healthcare Mater. (2023), 12, 2203233. These sensors can produce a signal correlated with the infection status of the wound fluid and/or tissues adjacent to the skin entry point. Said signal may be recognized by a caregiver by direct inspection of the dressing and/or recognized by remote reporting means, RF or otherwise. In embodiments of the present invention, the means of detection and signaling of infection status can detect pathogens illustratively including staph, MRSA, and Candida aureus.

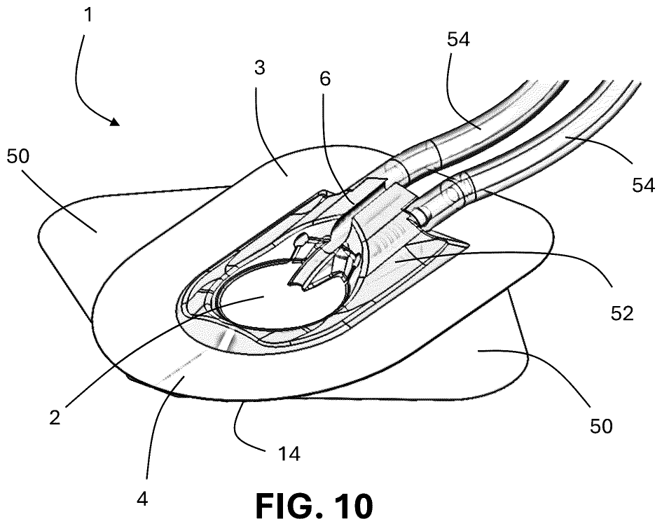

FIG. 10 shows a perspective view of a foundation dressing module according to embodiments prior to being placed at a skin entry point. As described above, the foundation dressing module 1 has a frame 3 defining an aperture 2. It is appreciated that the aperture 2 is configured to accommodate, straddle, and leave exposed an anticipated location of a skin entry site 8 of a vascular access catheter 9. The frame 3 has a foundation dressing non-skin facing surface 4 and a foundation dressing skin-facing surface 14. In some embodiments, the foundation dressing skin-facing surface 14 is backed with a skin-compatible adhesive. According to embodiments, the skin-facing surface 14 is initially covered with an adhesive release liner 50, which is configured to be removed to expose the adhesive layer of the skin-facing surface 14. According to embodiments, the foundation dressing module includes a rigid dressing body 52, which as shown in FIG. 10 optionally can include a plurality of tubes 54 for venting and application of a vacuum.

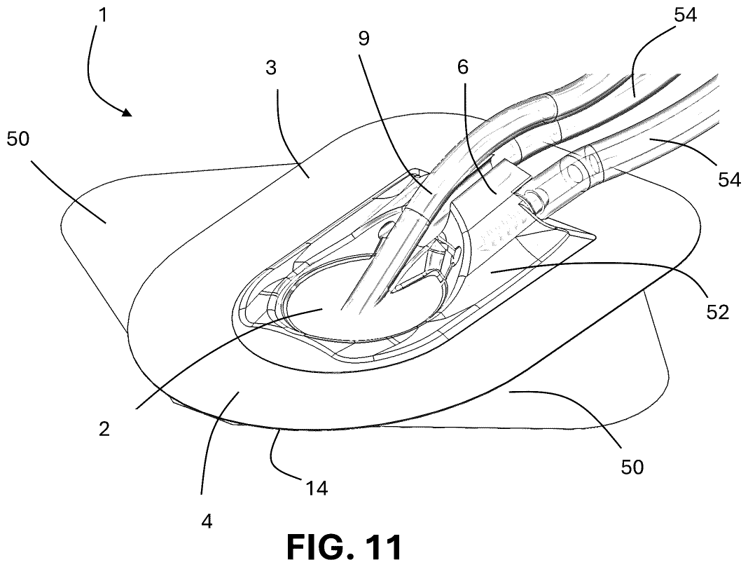

FIG. 11 shows a perspective view of a foundation dressing module 1 of FIG. 10 being placed at a skin entry point with the catheter 9 not yet fixed in the foundation dressing module 1. In FIG. 11, the catheter 9 has been threaded through the aperture 2 in the dressing and the release liner 50 is still attached to the skin-facing surface 14 of the foundation dressing module.

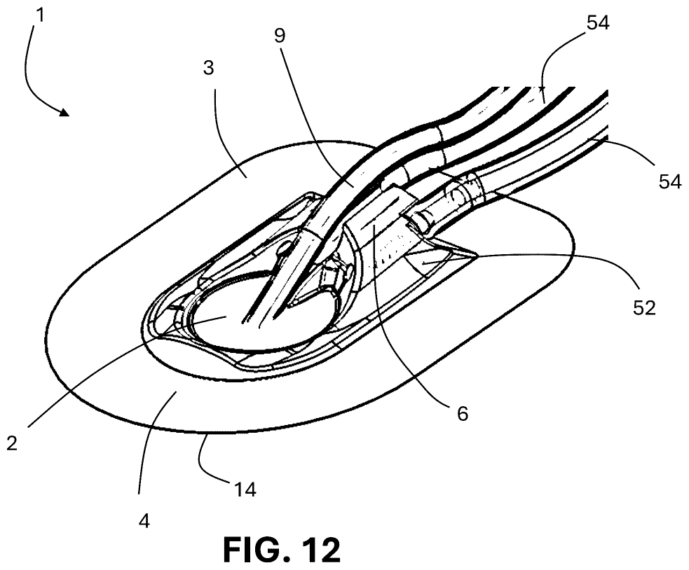

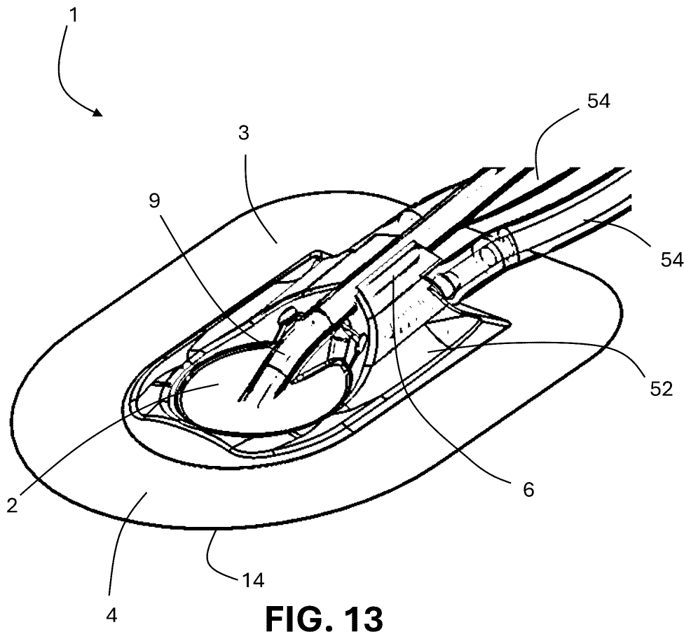

FIG. 12 shows a perspective view of the foundation dressing module 1 with the release liner 50 removed from the skin-facing surface 14 of the dressing to secure the dressing to the skin around the skin entry point. Then, in FIG. 13 the catheter 9 is gently pushed into the saddle 6 of the dressing, which according to embodiments is a channel. Care is taken to ensure that the catheter 9 is well seated and there are no folds near the lips of the channel.

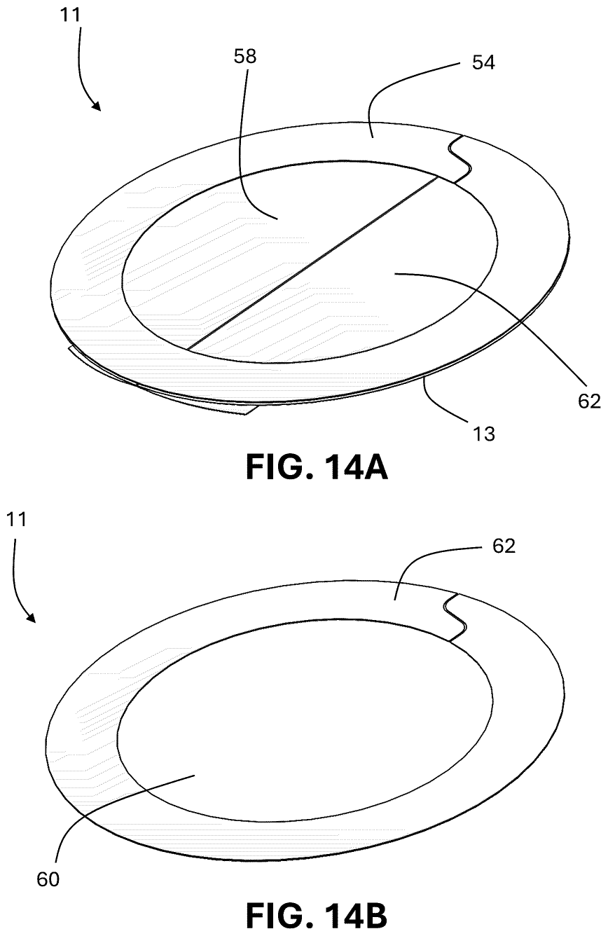

FIGS. 14A and 14B show a perspective view of a cover dressing module 11 for covering a foundation dressing module 1. The cover dressing module 11 has a cover dressing skin-facing surface 13 and a cover dressing non-skin facing surface 22. In FIG. 14A the cover dressing module 11 includes a removable release liner 58 applied to the skin-facing surface 13 prior to the release liners 58 being removed therefrom. In FIG. 14B, the release liners 58 have been removed therefrom to expose the adhesive of the skin-facing surface 13 of the cover dressing module 11. As shown in the figures, embodiments of the cover dressing module 11 include a transparent window 60.

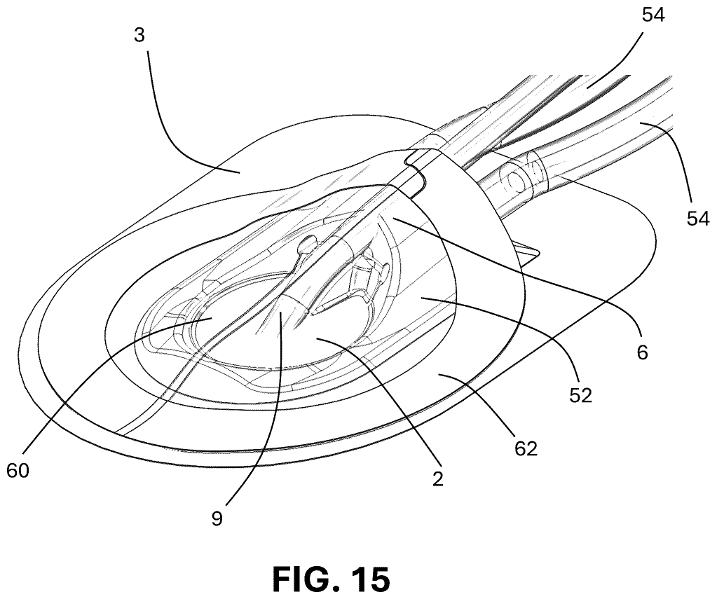

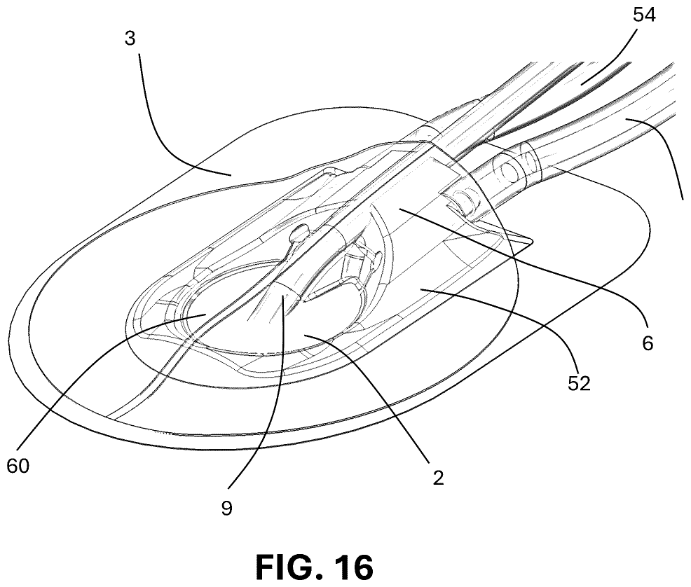

FIG. 15 shows a perspective view of the foundation dressing module 1 with the catheter 9 loaded into the saddle 6 and the cover dressing module 11 mounted thereto. As shown in FIG. 15, the window 60 is located such that it is concentric with the aperture 2 of the foundation dressing module 1. The window 60 is pressed against the dressing 1, starting at the catheter 9 and proceeding around the window 60. In FIG. 15 the cover dressing module 11 is shown with a removable window frame 62, which is provided in order to prevent wrinkles in the window 60. In FIG. 16, the window frame 62 has been removed from the cover dressing module 11.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A dressing for protecting a vascular access catheter skin entry site comprising:
a foundation dressing module comprising: a frame defining an aperture, said frame having a foundation dressing skin-facing surface and a foundation dressing opposing surface in opposition to the foundation dressing skin-facing surface; and
a cover dressing module comprising a cover dressing skin-facing surface and a cover dressing non-skin facing surface, a hydroscopic pad on the cover dressing skin-facing surface adapted to area-fill the aperture, the cover dressing skin-facing surface configured to be superimposable onto the foundation dressing opposing surface bringing the hydroscopic pad into direct fluid communication with a skin entry site of the vascular access catheter.

2. The dressing of claim 1 further comprising a frame extension adjacent to said frame and having a top surface and a bottom surface, a securement attached to the top surface of said frame extension configured to secure a vascular access catheter.

3. The dressing of claim 1 further comprising an adhesive on at least one of the foundation dressing opposing surface, the foundation dressing skin-facing surface, the top surface of said frame extension, the bottom surface of said frame extension, the cover dressing skin-facing surface, or a combination thereof.

4. The dressing of claim 1 wherein said securement further comprises a saddle and at least one adhesive strap.

5. The dressing of claim 1 wherein said securement stabilizes a vascular access catheter with: more than one catheter lumen, more than one intermediate hub, more than one terminal hub, or a combination thereof.

6. The dressing of claim 1 wherein said hydroscopic pad is a hydrogel, an absorbent gauze, a super absorbent polymer, a water-vapor-permeable membrane, or a combination thereof.

7. The dressing of claim 1 further comprising a fluid saturation detector configured to send a signal indicating a fluid saturation level of said hydroscopic pad.

8. The dressing of claim 1 further comprising an infection detector configured to send a signal indicating the presence of an infectious agent within the environment in and around the skin entry site of the vascular access catheter.

9. A dressing for protecting a vascular access catheter skin entry site at a non-planar body location comprising:
a foundation dressing module comprising a multi-part frame defining a groove, said multi-part frame having a foundation dressing skin-facing surface and a foundation dressing opposing surface opposing surface in opposition to the foundation dressing skin-facing surface, a frame extension adjacent to one part of said multi-part frame and having a top surface and a bottom surface, a securement attached to the top surface of said frame extension adapted to secure a vascular access catheter; and
a cover dressing module comprising a cover dressing skin-facing surface and a cover dressing non-skin facing surface, a hydroscopic pad on the cover dressing skin-facing surface configured to area-fill the groove, the cover dressing skin-facing surface configured to be superimposable onto the foundation dressing non-skin-facing surface bringing the hydroscopic pad into direct fluid communication with a skin entry site of the vascular access catheter.

10. The dressing of claim 9 wherein said cover dressing module floats above the skin entry site when attached to the foundation dressing non-skin-facing surface.

11. The dressing of claim 9 wherein said cover dressing module is longitudinally, latitudinally, azimuthally, torsionally flexible, or a combination thereof.

12. The dressing of claim 9 wherein said cover dressing module is formed from a folding pattern.

13. The dressing of claim 12 wherein the folding pattern is herringbone, accordion, yoshimura, miura, waterbomb, kresling, resich, moiré, or a combination thereof.

14. The dressing of claim 9 further comprising an adhesive on the foundation dressing non-skin-facing surface, the foundation dressing skin-facing surface, the top surface of said frame extension, the bottom surface of said frame extension, the cover dressing skin-facing surface, or a combination thereof.

15. The dressing of claim 9 wherein said securement further comprises a saddle and at least one adhesive strap.

16. The dressing of claim 9 wherein said securement stabilizes a vascular access catheter with more than one catheter lumen, more than one intermediate hub and more than one terminal hub.

17. The dressing of claim 9 wherein said hydroscopic pad is a hydrogel, an absorbent gauze, a super absorbent polymer, a water-vapor-permeable membrane, or a combination thereof.

18. The dressing of claim 9 further comprising a fluid saturation detector configured to send a signal indicating a fluid saturation level of said hydroscopic pad.

19. The dressing of claim 9 further comprising an infection detector configured to send a signal indicating the presence of an infectious agent within the environment in and around the skin entry site of the vascular access catheter.

20. A method of protecting an environment in and around a skin entry site of a vascular access catheter comprising:
placing a dressing of claim 1 on skin around the skin entry site.
